(19) United States
(12) Patent Application Publication
(10) Pub. No.: US 2006/0229826 A1
(43) Pub. Date: Oct. 12, 2006

(54) Quantitative structure - activity relationships (QSAR)

(76) Inventors: Evgueni Kolossov, Berkshire (GB); Robert Stanforth, Surrey (GB)

Correspondence Address: FULBRIGHT AND JAWORSKI LLP, 555 S. FLOWER STREET, 41ST FLOOR, LOS ANGELES, CA, 90071, US

(21) Appl. No.: 11/388,741

(22) Filed: Mar. 24, 2006

(30) Foreign Application Priority Data
Mar. 30, 2005 (GB) 0506366.4

(51) Int. Cl. G06F 19/00; C12M 1/34

(52) U.S. Cl. 702/19; 702/20; 435/287.2

(57) ABSTRACT

A method for estimation of the distance from a domain by means of a fragment-based model, the method comprising the steps of identifying the fragments [r₁ to rₙ] in a structure, comparing the or each fragment with one or more fragments in the model, if the or each fragment substantially matches a fragment in a model determining a first error measure between a contribution of the or each fragment and a contribution of the matching fragment in the model, if the or each fragment does not substantially match a fragment in the model determining which fragment in the model is the most similar to the or each fragment and determining a second error measure based on the similarity between the fragment and the most similar fragment; and combining the first error measure and the second error measure to generate a degree of separation between the activity of the structure and of the combined contribution of the fragments in the model.

### FIELD OF THE INVENTION

This invention relates to Quantitative Structure activity relationships (QSAR) in particular, but not exclusively to the development and validation status of QSAR models.

### BACKGROUND OF THE INVENTION

QSAR represents a technique to model the relationships between the chemical structures and their biological or physico-chemical properties. The models and correlation techniques are used to predict in silico what properties a chemical structure might have. This enables new chemicals and drugs to be created which are more likely to meet certain criteria. For example, the prediction of a bad side effect can prevent a potential drug from the next stage of research and in this way, save money and resources. There are clearly many advantages in using this type of analysis to speed up the creation and/or manufacture of new chemicals and prevent potential drug failures at earlier stages of the creation or manufacture.

Some QSAR models are created using fragments or bits of a specific chemical compound. For example the fragments may be groups attached to say an organic compound such as: a nitro group, an ethyl group, etc. The fragments once identified are compared with fragments in the QSAR fragment model to determine a degree of correlation between the fragments and the activity.

A QSAR fragment-based model is the set of fragments and their contributions (weights). To predict the property/activity for the structure in question, this structure needs to be split into the fragments. Then for each of the fragments the match needs to be found in the set of fragments in the model. The final prediction value for this structure will be the sum of contributions for all fragments. If there is no match in the model for a particular fragment, then the prediction actually fails. This problem in QSAR modelling is called ‘problem of missing fragments’.

At present there are no clearly defined universal quantitative determinations to enable consistent regulatory principles for QSAR models. This is an issue which is currently under consideration by many, but particularly the Organisation for Economic Co-operation and Development (OECD). The main principles known as the Setubal principles are that a QSAR should:

a. have a defined endpoint;

b. take the form of an unambiguous algorithm;

c. have a defined domain of applicability;

d. have appropriate measure of goodness-of-fit, robustness and predictivity; and

e. have a mechanistic interpretation, if possible.

The principle which is causing the most problems is the principle of having a defined domain of applicability. The need to define an applicability domain takes account of the fact that QSARs are reductionist models and generally have a number of limitations. These limitations are in terms of the types of chemical structures, physicochemical properties and mechanisms of action for which the model can generate reliable predictions. It is thus an object to define the type of information necessary to define QSAR applicability domains and how to determine this.

A QSAR descriptor-based model is the set of descriptor contributions (weights). To predict a property/activity for the structure in question descriptor values are calculated and multiplied by the contribution values. Because the descriptor values are numerical, it is possible to have a definition for the domain: For example, if there is only one descriptor, then any value calculated for the structure in question which is lower than the lowest values or higher than the highest values (all structures in a training set equal the model) will indicate that this compound is outside the domain. The distance from the domain will be the distance between the values calculated for the structure in question and the lowest or highest value in the model. This approach cannot be used for fragment-based models because fragments unlike the descriptors are not numerical.

For descriptor-based models a set of well known methods can be applied for domain of applicability assessment. An example of this is disclosed in a paper by R. S. Sheridan et al. (See: R. S Sheridan, B P Fenston, V N Maiorov and S K Kearsley; Similarity of molecules in the training set is a good discriminator for prediction accuracy in QSAR. J. Chem. Inf. Comput.SCI., 2004(44), 1912-1928). In this method the most useful measures that were tested are:

1. The similarity of the molecule to be predicted to the nearest molecule in the training set; and/or

2. The numbers of neighbours in the training set, where neighbours are those more similar than a user chosen cut-off.

However fragment-based models tend to be more significant and produce more information for research than descriptor-based models, as previously identified. The methods identified above do not however work for fragment based models for the following reasons:

a. no account is taken of the number of fragments generated from the structure;

b. no account is taken of any similarities between fragments generated from the structure and fragments in the model; and

c. no measure of any distance from the domain is taken to indicate degree of error in any predictions.

One object of the present invention is to provide a method of determining domain of applicability for fragment-based QSAR model.

Another object of the present invention is to overcome at least some of the disadvantages of know QSAR modelling.

### SUMMARY OF THE INVENTION

A method for estimation of the distance from a domain by means of a fragment-based model, the method comprising the steps of:
identifying the fragments [r₁ to rₙ] in a structure;
comparing the or each fragment with one or more fragments in the model;
if the or each fragment substantially matches a fragment in a model determining a first error measure between a contribution of the or each fragment and a contribution of the matching fragment in the model;
if the or each fragment does not substantially match a fragment in the model determining which fragment in the model is the most similar to the or each fragment and determining a second error measure based on the similarity between the fragment and the most similar fragment; and
combining the first error measure and the second error measure to generate a degree of separation between the activity of the structure and of the combined contribution of the fragments in the model.

Preferably the step of identifying the fragments (r) further comprises determining a mapping count X of the number of times fragment (r) in the structure (k) such that
X=[mₖᵣ].

Advantageously the step of determining the first error measure comprises calculating an error weight coefficient eᵣ in accordance with

$$\begin{matrix}{e}_{r} = {E}_{rr} & 4 \\ where E = {( {X}^{T} X - \frac{1}{N} ( {\sum }_{k = 1}^{N} {x}^{k} ) ( {\sum }_{k = 1}^{N} {x}_{k}^{T} ) )}^{- 1} & 5\end{matrix}$$

N is the number of structures which have been trained on in the model; and
xₖ^{T}=[mₖᵣ] is the row of X containing the mappings for structure k.

Preferably the step of determining the second error measure comprises measuring the degree of dissimilarity (1−T) of the or each fragment from the most similar fragment in the model.

In one embodiment the step of combining comprises combining the error weight coefficient and the dissimilarity.

Preferable the distance from the domain is estimated from the step of combining the first and second error measurements and the distance from the domain is estimated in accordance with:

$${d}_{fm}^{2} = \frac{1}{m} {\sum }_{r = 1}^{m} ( 1 - {T}_{r} ) {m}_{r}^{2} {e}_{r}$$

The above techniques can also be used in a method of determining the match between a structure and a model of a structure.

According to a second aspect of the present invention there is provided Apparatus for estimating the distance from a domain comprising:
identification means for identifying the fragments [r₁ to rₙ] in the structure;
comparison means for comparing the or each fragment with one or more fragments in a model;
if the or each fragment substantially matches a fragment in the model, a first determination means for determining a first error measure between a contribution of the or each fragment and a contribution of the matching fragment in the model;
if the or each fragment does not substantially match a fragment in the model, a second determination means for determining which fragment in the model is the most similar to the or each fragment and determining a second error measure based on the similarity between the fragment and the most similar fragment; and
combining means for combining the output from the first and second determining means to generate a degree of separation between the activity of the structure and of the combined contribution of the fragments in the model.

Preferably the second measuring means comprises measuring means for measuring the degree of dissimilarity (1−T) of the or each fragment from the most similar fragment in the model.

Also preferably the apparatus further comprises mapping means for determining a mapping X of the fragments in the structure such that: X=[mₖᵣ].

The above defined method and apparatus allow the calculation of a prediction even if there is the problem of missing fragments and allows a level of uncertainty (error) to be determined for the prediction. Other advantages are identified in the description.

### DESCRIPTION OF THE DRAWINGS

Reference will now be made, by way of example, to the accompanying drawings in which:

FIG. 1 is a diagram of an example of a chemical structure showing a number of fragments which is used to illustrate certain facets of the invention,

FIG. 2 is a diagram showing the structures for two specific molecules (7-Methylthiopteridine and Fenitrothion),

FIG. 3 is a table showing the contribution of fragments generated from the molecule of 7-Methylthiopteridine,

FIG. 4 is a table showing the contribution of fragments generated from the molecule of Fenitrothion,

FIG. 5 is a diagram showing the original and replacement fragments for 7-Methylthiopteridine,

FIG. 6 is a diagram showing the original and replacement fragments for Fenitrothion.

### DETAILED DESCRIPTION OF THE INVENTION

QSARs are a mathematical relationship linking chemical structure and properties of that chemical in a quantitative manner for a series of compounds.

Methods such as regression and pattern recognition techniques are often adopted. Regression analysis is the use of statistical methods for modelling a set of dependant variable in terms of combinations of predictors. Methods such as multiple linear regression (MLR) and partial least squares (PLS) are included.

Pattern recognition is the identification of patterns in large data sets using mathematical analysis.

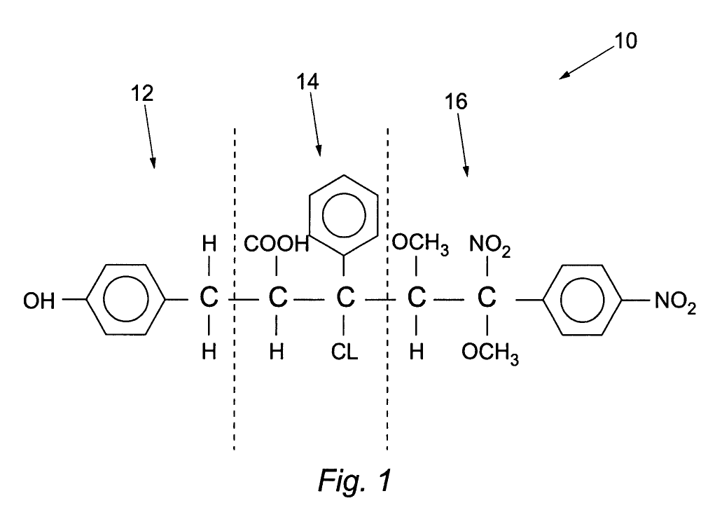

Referring initially to FIG. 1, a structure k (10 in FIG. 1) is shown. The structure has an experimentally determined activity (y). Mathematically this is represented as:
y=[yₖ]. . . 1

In addition the structure k (10) has a number of fragments r (12, 14, 16 in FIG. 1). The number of times a specific fragment r occurs in a structure k is given by the mapping count X of the fragments. Mathematically this may be expressed as:
X=[mₖᵣ]. . . 2

A multivariant linear regression process is used to model the activity.

$$Y \approx {a}_{0} + {\sum }_{r} {a}_{r} {m}_{r} \qquad 3$$

Where aₒ is a constant and aᵣ is the contribution for the variable mᵣ and mᵣ varies over the m fragments. The values of aₒ and aᵣ are determined that best fit the data.

The regression calculation also produces an “error weight” coefficient eᵣ for each fragment r, from the diagonal of the inverse of the centred matrix. This can be expressed mathematically as:

$$\begin{matrix}{e}_{r} = {E}_{rr} & 4 \\ where \text{:} \\ E = {( {X}^{T} X - \frac{1}{N} ( {\sum }_{k = 1}^{N} {x}^{k} ) ( {\sum }_{k = 1}^{N} {x}_{k}^{T} ) )}^{- 1} & 5\end{matrix}$$

N is the number of structures which have been trained on in the model; and
xₖ^{T}=[mₖᵣ] is the row of X containing the mappings for structure k.

The QSAR model has a statistical variance σ² and the coefficient eᵣ for each fragment is an indication of statistical variance in aᵣ relative to that of the model. Thus it can be concluded that a mapping count of mᵣ for fragment r will contribute the following to the statistical variance of the predicted activity of the structure:
mᵣ²eᵣσ². . . 6

It should be noted that this is a somewhat simplified contribution which does not take into account correlation between different fragments' mapping. The contribution is intended to indicate the isolated effect of a single fragment. In the present invention this statistical variance is an important feature as it is representative of a “distance from the model” of a specific fragment. This is so as a high value of eᵣ corresponds to only a little variation of the data set with respect to mᵣ.

When applying a fragment type QSAR model to a new structure there is a good chance that some (and maybe all) of the fragments are not present in the model. In this case a fragment in the structure is compared to a fragment in the model which is the most similar to it. The similarity is based on certain atom/bond configurations and may be expressed in terms of a coefficient, for example the Tanimoto coefficient T. The Tanimoto coefficient is a measure of similarity and has a value between 0 and 1 or 0% and 100%, depending on preference. Different measures of similarity can be used and the method of calculation is independent of the methods of similarity measurement.

The dissimilarity (1−T) of a fragment relative to the most similar fragment in the model, gives rise to another source of uncertainty. This uncertainty can also be interpreted as a “distance from the model” for a specific fragment.

This invention presents a means by which the two above mentioned uncertainties are combined for all fragments of a new structure to provide a single measure d_{fm}. This measure d_{fm} is a measure of the distance of the structure from the domain of the fragment model. For a fragment-based model domain is the set of fragments. In a preferred method the measure can be determined from combining the “error weight” uncertainty and the dissimilarity uncertainty. This can be represented mathematically as:

$${d}_{fm}^{2} = \frac{1}{m} {\sum }_{r = 1}^{m} ( 1 - {T}_{r} ) {m}_{r}^{2} {e}_{r} \qquad 7$$

Where the sum is for all m fragments occurring in the structure and Tᵣ is the similarity of fragment r in the structure to its closest match in the model.

The expression in equation 7 relates to a “squared distance measure” as it involves squared factors m² and E. This is consistent with how regression measures r² and q² are used to determine standard Euclidian distance in other areas of statistical analysis.

The method described above provides in non mathematical terms that only fragments that are not in the model contribute to d_{fm}²; or mathematically if the similarity is equal to 1 (i.e. the fragment is the same) then this element of the equation will be zero (1−1=0), so will not contribute to the distance. If all fragments of a particular structure are in the model then the value of d_{fm}² will inevitably be zero.

The above method provides a method in which there is a clear definition of a domain of applicability for fragment-based QSAR models and the distance (or degree of error) from the domain is clearly identified.

This will mean that fragment-based models will provide a far greater degree of certainty in the determination of the properties of a chemical structure which is analysed by this type of fragment based model. This in turn will mean that chemists wishing to design and generate new chemical structures for whatever purpose will have a more accurate means of prediction.

The method described herein suggests a set of mathematical representations all of which are by way of example only. Other ways of determining the error weight uncertainty for each fragment and of determining the dissimilarity uncertainty of each fragment and for combining them are equally valid, as will be appreciated by those skilled in the art.

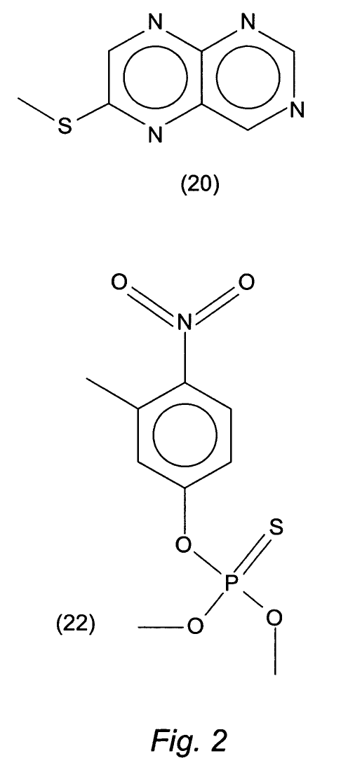

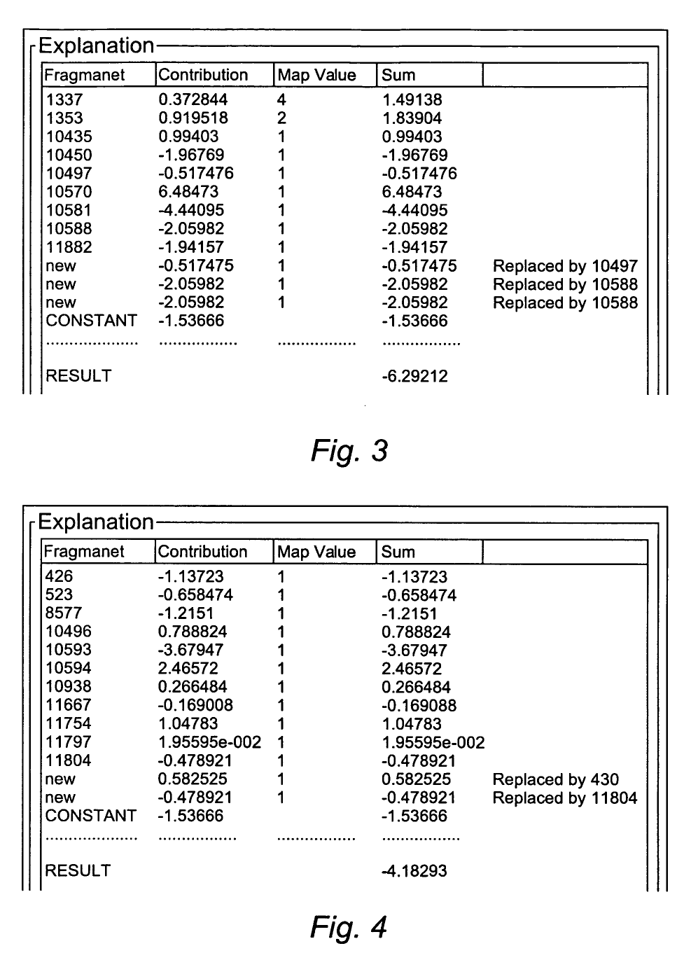

An example of use of the model is now described. During the external test set validation process using a fragment-based model for two molecules, not all fragments were found in the model. The structures for these molecules are represented in FIG. 2. Calculated values for both compounds based on the summary of contribution for all fragments generated from the molecules are shown in the tables in FIGS. 3 and 4.

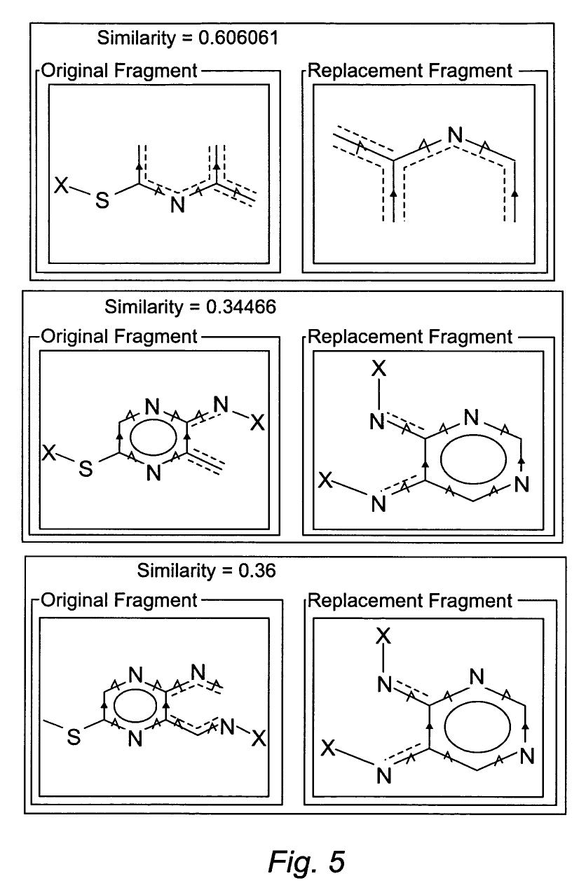

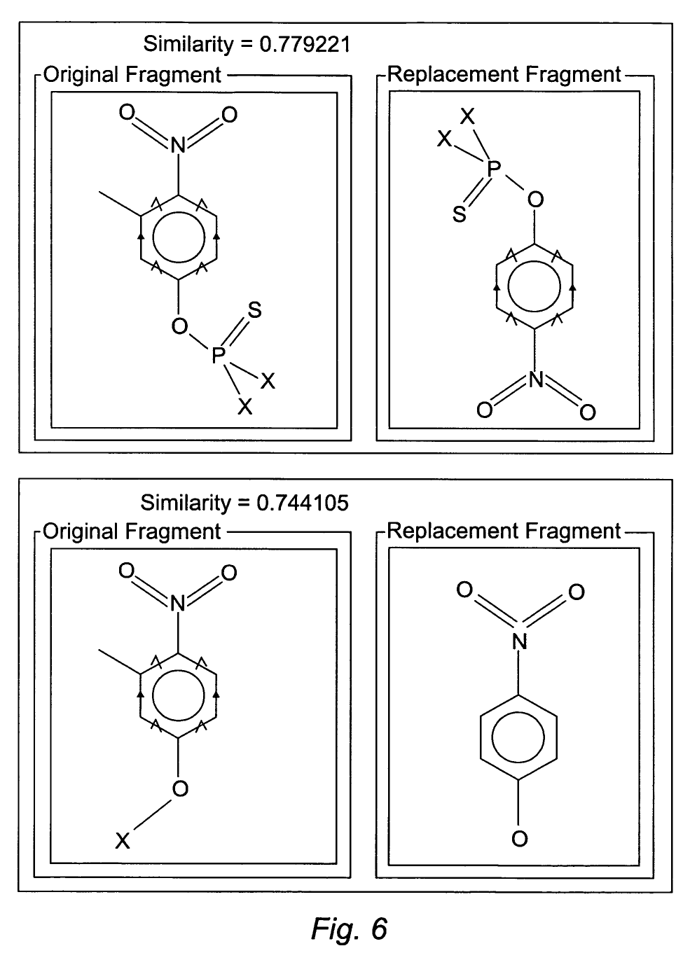

Both structures contain approximately the same number of fragments (12 and 13). For 7-Methylthiopteridine there are three fragments, which were not found in the model and have been replaced with the most similar fragment from the model. For Fenitrothion there are two such fragments. The original fragments and replacements are presented in FIGS. 5 and 6 respectively.

It is important to now determine what prediction can be trusted and which can not. To determine this it is necessary to calculate the distance from the domain using formula (7) based on the statistics presented below in Table 1.

TABLE 1

| | | Replacement | Similarity to | | | | |
|---|---|---|---|---|---|---|---|
| | Num. of | Fragments | the original | Mapping | Error | | |
| Compound | Fragments | ID | fragment | value | Weight | Contribution | dfm² |
| 7-Methylthi- | 12 | 10497 | 0.606061 | 1 | 1.351 | 0.532211 | |
| opteridine | | 10588 | 0.34466 | 1 | 167.9 | 110.0315 | |
| | | 10588 | 0.36 | 1 | 167.9 | 107.456 | |
| | | | | | | 218.0197 | 18.16832 |
| Fenitrothion | 13 | 430 | 0.744186 | 1 | 0.768 | 0.196465 | |
| | | 11804 | 0.779221 | 1 | 2.122 | 0.468493 | |
| | | | | | | 0.664958 | 0.051151 |

The values of d_{fm}² are calculated below as follows:

$$\begin{matrix}\begin{matrix}{d}_{fm}^{2} ( 7 - Methylthiopteridine ) = ( 1 / 12 ) * [ ( 1 - 0.606061 ) * {1}^{2} * \\ 1.351 + ( 1 - 0.34466 ) * {1}^{2} * 167.9 + \\ ( 1 - 0.36 ) * {1}^{2} * 167.9 ] \\ = ( 1 / 12 ) * [ 0.5322 + 110.0 + 107.5 ] \\ = 218.0 / 12 \\ = 18.17\end{matrix} \\ \begin{matrix}{d}_{fm}^{2} ( Fenitrothion ) = ( 1 / 13 ) * [ ( 1 - 0.744186 ) * {1}^{2} * 0.7680 + \\ ( 1 - 0.779221 ) * {1}^{2} * 2.122 ] \\ = ( 1 / 13 ) * [ 0.1965 + 0.4685 ] \\ = 0.6650 / 13 \\ = 0.05115\end{matrix}\end{matrix}$$

Comparison between these two values (18.17 and 0.05115) provides the answer: Fenitrothion is very close to the domain of the model whilst 7-Methylthiopteridine is very far from this domain. The prediction value for the 7-Methylthiopteridine cannot therefore be taken in account. This result corresponds to the experimental values/residuals for these structures:

7-Methylthiopteridine: Experimental value is −1.55, predicted value is −6.292, and the absolute residual value is 4.742 (305.9%). Fenitrothion: Experimental value is −4.04, predicted value is −4.183, and the absolute residual value is 0.1429 (3.54%).

The new simple and quantitative method for assessment of domain of applicability for the fragment-based QSAR models is thus proposed. The model takes into account the total number of fragments generated from the structure, availability the most similar fragments in the model and their contributions values. Implementation of this method allows the definition of statistical significance of the predictions and solving the problem of missing fragments, which is crucial for fragment-based QSAR models.

1. A method for estimation of the distance from a domain by means of a fragment-based model, the method comprising the steps of:
identifying the fragments [r₁ to rₙ] in a structure;
comparing the or each fragment with one or more fragments in the model;
if the or each fragment substantially matches a fragment in a model determining a first error measure between a contribution of the or each fragment and a contribution of the matching fragment in the model;
if the or each fragment does not substantially match a fragment in the model determining which fragment in the model is the most similar to the or each fragment and determining a second error measure based on the similarity between the fragment and the most similar fragment; and
combining the first error measure and the second error measure to generate a degree of separation between the activity of the structure and of the combined contribution of the fragments in the model.

2. The method of claim 1, wherein the step of identifying the fragments (r) further comprises determining a mapping count X of said fragments in the structure such that
X=[mₖᵣ].

3. The method of claim 1, wherein the step of determining the first error measure comprises calculating an error weight coefficient eᵣ in accordance with
$$\begin{matrix}{e}_{r} = {E}_{rr} & 4 \\ where E = {( {X}^{T} X - \frac{1}{N} ( {\sum }_{k = 1}^{N} {x}^{k} ) ( {\sum }_{k = 1}^{N} {x}_{k}^{T} ) )}^{- 1} & 5\end{matrix}$$
N is the number of structures which have been trained on in the model; and
xₖ^{T}=[mₖᵣ] is the row of X containing the mappings for structure k.

4. The method of claim 1, wherein the step of determining the second error measure comprises measuring the degree of dissimilarity (1−T) of the or each fragment from the most similar fragment in the model where T is a similarity constant.

5. The method of claim 4, wherein the step of combining comprises combining the error weight coefficient and the degree of dissimilarity.

6. The method of claim 1, wherein the distance from the domain is estimated from the step of combining the first and second error measurements.

7. The method of claim 1, wherein the distance from the domain is estimated in accordance with:
$${d}_{fm}^{2} = \frac{1}{m} {\sum }_{r = 1}^{m} ( 1 - {T}_{r} ) {m}_{r}^{2} {e}_{r}$$

8. A method of determining the match between a structure and a model of a structure including:
a method for estimation of the distance from a domain by means of a fragment-based model, the method comprising the steps of:
identifying the fragments [r₁ to rₙ] in a structure;
comparing the or each fragment with one or more fragments in the model;
if the or each fragment substantially matches a fragment in a model determining a first error measure between a contribution of the or each fragment and a contribution of the matching fragment in the model;
if the or each fragment does not substantially match a fragment in the model determining which fragment in the model is the most similar to the or each fragment and determining a second error measure based on the similarity between the fragment and the most similar fragment; and
combining the first error measure and the second error measure to generate a degree of separation between the activity of the structure and of the combined contribution of the fragments in the model.

9. Apparatus for estimating the distance from a domain comprising:
identification means for identifying the fragments [r₁ to rₙ] in the structure;
comparison means for comparing the or each fragment with one or more fragments in a model;
if the or each fragment substantially matches a fragment in the model, a first determination means for determining a first error measure between a contribution of the or each fragment and a contribution of the matching fragment in the model;
if the or each fragment does not substantially match a fragment in the model, a second determination means for determining which fragment in the model is the most similar to the or each fragment and determining a second error measure based on the similarity between the fragment and the most similar fragment; and
combining means for combining the output from the first and second determining means to generate a degree of separation between the activity of the structure and of the combined contribution of the fragments in the model.

10. The apparatus of claim 9, wherein the second measuring means comprises measuring means for measuring the degree of dissimilarity (1−T) of the or each fragment from the most similar fragment in the model.

11. The apparatus of claim 9, further comprises mapping means for determining a mapping X of the fragments in the structure such that X=[mₖᵣ].

12. Apparatus for estimating the distance from a domain comprising an identifier which can identify one or more fragments [r₁ to rₙ] in a structure;
a comparator which can compare the or each fragment with one or more fragments of a model 1;
a first measuring device which can evaluate a first error measure between a contribution of the or each fragment and a contribution of the matching fragment in the model if the or each fragment substantially matches a fragment of the model;
a second measuring device which can determine which fragment in the model is the most similar to the or each fragment in order to determine a second error measure based on the similarity, if the or each fragment does not substantially match a fragment of the model;
a third measuring device which can combine the first and second error measures to produce a degree of separation between the activity of the structure and of the combined contribution of the fragments in the model.
